(19) United States
(12) Patent Application Publication
(10) Pub. No.: US 2020/0060604 A1
(43) Pub. Date: Feb. 27, 2020

(54) SYSTEMS AND METHODS OF AUTOMATIC COUGH IDENTIFICATION

(71) Applicant: HOLLAND BLOORVIEW KIDS REHABILITATION HOSPITAL, Toronto (CA)

(72) Inventors: Helia Mohammadi, Toronto (CA); Tom Chau, Toronto (CA)

(21) Appl. No.: 16/487,180

(22) PCT Filed: Feb. 22, 2018
(86) PCT No.: PCT/CA2018/050203

§ 371 Date: Aug. 20, 2019

Related U.S. Application Data
(60) Provisional application No. 62/463,301, filed on Feb. 24, 2017.

(51) Int. Cl. A61B 5/00; A61B 5/08; A61B 5/11

(52) U.S. Cl. CPC A61B 5/4205; A61B 5/0823; A61B 5/7267; A61B 5/4803; A61B 5/11; A61B 2503/08; A61B 5/7405; A61B 5/742; A61B 5/0002; A61B 5/6822; A61B 2562/0219; A61B 5/7207

(57) ABSTRACT

A method can use dual-axis accelerometry signals obtained during a time period to classify segments of the time period as a cough or as a non-cough artifact (e.g., a rest state, a swallow, a tongue movement, or speech). The method can include representing segments of the dual-axis accelerometry signals as meta-features for each segment of the time period, preferably one or more time features, frequency features, time-frequency features, or information-theoretic features for each segment. The salient meta-features can be used to classify the segments as a cough or a non-cough artifact. Preferably a processing module operatively connected to the sensor performs the processing of the dual-axis accelerometry signals and also automatically classifies the segments. The method and/or the device can be used to diagnose or treat a dysphagia patient, for example by discriminating a cough from a swallow.

### BACKGROUND

The present disclosure generally relates to identifying a cough. More specifically, the present disclosure relates to an automatic cough detection and monitoring system that discriminates cough accelerometry signals from other artifacts such as rest state, swallowing, head movements, and speech.

A cough is a protective mechanical response in which rapid contractions of the thoracic cavity generate a forceful and rapid expulsion of air that clears the airway of foreign material, fluid or mucus. Cough can be symptomatic of various respiratory conditions such as asthma, rhinitis and gastro-oesophageal reflux disease in adults and protracted bronchitis in children. Cough is also a normal reflexive response to aspiration, which is the entry of foreign material into the airway seen in people with swallowing difficulties. Hence, knowledge of cough severity, including intensity and frequency, may inform clinical decision-making in terms of appropriate treatment of the underlying issue. However, clinical assessments of cough often involve subjective judgment of symptoms and symptom severity, leading to inconsistent symptom reports between patients and caregivers. Cough scores, diaries, symptom questionnaires and visual analogue scales generally lack validation as tools for evaluating cough severity.

Currently, there are a number of commercially available cough monitoring devices. Generally, these microphone-based systems are unable to distinguish true coughs from ambient noise and non-cough patient sounds, and the performance of a commercial cough monitor in a comparative analysis was inconsistent across subjects (Drugman et al., “Objective study of sensor relevance for automatic cough detection,” Biomedical and Health Informatics, IEEE J. Biomed. Health Inform. 17(3):699-707 (2013)). In a recent validation against manually identified coughs, another commercial cough detector yielded low sensitivity (Turner et al., “How to count coughs? Counting by ear, the effect of visual data and the evaluation of an automated cough monitor,” Respir. Med. 108(12):1808-1815 (2014)). The development of a fully automated, accurate cough monitoring system remains an elusive challenge.

To circumvent some of the above limitations, recent research on automatic cough detection has invoked multiple sensors. For example, Drugman et al. (cited above) compared six different sensors against a commercial cough monitor and found that an omnidirectional lapel microphone was the most sensitive to coughs. Turner et al. (cited above) compared the counts of coughs detected by human experts against those identified by a sensor combination consisting of thoracic respiratory belt and tracheal and chest microphones. Recently, Hirai et al. used a microphone (over the second intercostal muscle) and an accelerometer (positioned over the abdomen) to count the number of overnight cough (“A new method for objectively evaluating childhood nocturnal cough,” Pediatr. Pulmonol., 50(5):460-468 (2015)).

### SUMMARY

The present inventors recognized that multi-transducer approaches have produced promising results but nevertheless require careful sensor positioning and attachment. Further, most of these approaches still retain a microphone, precluding their use in noisy environments. The present inventors recognized that an alternative approach may be to exclusively deploy a sensor, such as an accelerometer, that is insensitive to ambient acoustic noise. As a result, disclosed herein are embodiments of a framework for detection of cough and non-cough events, preferably using dual-axis accelerometry signals from a single accelerometer on the patient's neck.

Accordingly, in a general embodiment, the present disclosure provides a method of identifying a cough. The method comprises: receiving, on a processing module, dual-axis accelerometry signals obtained by a sensor positioned externally on an anterior-posterior (A-P) axis and a superior-inferior axis (S-I) of the throat of a subject; representing segments of the dual-axis accelerometry signals as meta-features comprising salient meta-features, the processing module performs the representing of the segments; and classifying the segments as one of a plurality of classifications comprising at least one classification that is a cough and at least one classification that is a rest state, the processing module performs the classifying based on the salient meta-features.

In an embodiment, at least one of the salient meta-features for each of the A-P axis and the S-I axis is selected from the group consisting of time domain characteristics of the accelerometry signals, information theoretic domain characteristics of the accelerometry signals, frequency domain characteristics of the accelerometry signals, and time-frequency domain characteristics of the accelerometry signals.

In an embodiment, at least one of the salient meta-features is selected from the group consisting of mean S-I, Lempel-Ziv complexity S-I, maximum energy A-P, variance A-P, and skewness A-P.

In an embodiment, the classifying of the segments comprises applying at least one of an artificial neural network (ANN) or a support vector machine (SVM) to the salient meta-features.

In an embodiment, the plurality of classifications comprises an additional classification that is at least one non-cough artifact selected from the group consisting of a swallow, a tongue movement, and speech. The at least one non-cough artifact preferably comprises a swallow.

In an embodiment, the sensor is a single dual-axis accelerometer, and the method is performed without using a microphone, a video recorder, or another accelerometer.

In an embodiment, the method comprises pre-processing of the dual-axis accelerometry signals before the representing of the segments of the dual-axis accelerometry signals as the meta-features, the pre-processing comprising at least one step selected from the group consisting of de-noising, head movement suppression, and high frequency noise filtering by wavelet packet decomposition.

In an embodiment, the plurality of classifications comprise at least one classification that is a voluntary cough and at least one classification that is an involuntary cough, and the method comprises discriminating between voluntary cough and involuntary cough.

In another embodiment, the present disclosure provides an apparatus for identifying a cough. The apparatus comprises: a sensor configured to be positioned on the throat of a patient and acquire vibrational data for an anterior-posterior axis and a superior-inferior axis; and a processing module operatively connected to the sensor and configured to represent segments of the dual-axis accelerometry signals as meta-features comprising salient meta-features used by the processing module to classify the segments as one of a plurality of classifications comprising at least one classification that is a cough and at least one classification that is a rest state.

In an embodiment, the apparatus comprises an output component selected from a display, a speaker, and a combination thereof, the processing module configured to use the output component to indicate the classification of the segments visually and/or audibly.

In an embodiment, the processing module is operatively connected to the sensor by at least one of a wired connection or a wireless connection.

In another embodiment, the present disclosure provides a method of diagnosing the presence or absence of coughing in a patient. The method comprises: positioning a sensor externally on the throat of the patient, the sensor acquiring vibrational data for at least one axis selected from the group consisting of an anterior-posterior axis and a superior-inferior axis, the sensor operatively connected to a processing module configured to represent segments of the dual-axis accelerometry signals as meta-features comprising salient meta-features used by the processing module to classify the segments as one of a plurality of classifications comprising at least one classification that is a cough and at least one classification that is a rest state; and treating the patient based on the classification of the segments.

In an embodiment, the method comprises determining a cough frequency based at least partially on the classification of the segments, and the treating of the patient is based at least partially on comparison of the cough frequency to a threshold.

In an embodiment, the patient is being evaluated for at least one medical condition selected from the group consisting of asthma, rhinitis, gastro-oesophageal reflux disease, bronchitis, and dysphagia.

In another embodiment, the present disclosure provides a method of diagnosing or treating dysphagia in a patient. The method comprises: positioning a sensor externally on the throat of the patient, the sensor acquiring vibrational data for at least one axis selected from the group consisting of an anterior-posterior axis and a superior-inferior axis, the sensor operatively connected to a processing module configured to represent segments of the dual-axis accelerometry signals as meta-features comprising salient meta-features used by the processing module to classify the segments as one of a plurality of classifications comprising at least one classification that is a cough and at least one classification that is a swallow.

In an embodiment, the patient has dysphagia, and the method further comprises adjusting treatment of the patient based at least partially on the classification of the segments. The adjusting of the treatment can comprise adjusting a feeding administered to the patient, and the adjusting of the feeding is selected from the group consisting of: changing a consistency of the feeding, changing a type of food in the feeding, changing a size of a portion of the feeding administered to the patient, changing a frequency at which portions of the feeding are administered to the patient, and combinations thereof.

In another embodiment, the present disclosure provides an apparatus for diagnosing or treating dysphagia. The apparatus comprises: a sensor configured to be positioned on the throat of a patient and acquire vibrational data for an anterior-posterior axis and a superior-inferior axis; and a processing module operatively connected to the sensor and configured to represent segments of the dual-axis accelerometry signals as meta-features comprising salient meta-features used by the processing module to classify the segments as one of a plurality of classifications comprising at least one classification that is a cough and at least one classification that is a swallow.

An advantage of one or more embodiments provided by the present disclosure is a fully automated, accurate cough monitoring system.

Another advantage of one or more embodiments provided by the present disclosure is to overcome drawbacks of known techniques for cough detection.

A further advantage of one or more embodiments provided by the present disclosure is to reject ambient noise, accommodate variation in the characteristics of coughs across individuals and conditions, and provide the capability to monitor the patient over a long period of time, especially during the night when self-reporting is not feasible.

Yet another advantage of one or more embodiments provided by the present disclosure is to consider both voluntary and reflexive coughs.

Another advantage of one or more embodiments provided by the present disclosure is a cough detection method that requires only a single accelerometer, in contrast to current cough monitoring systems which require combinations of microphones, accelerometers, and video recorders.

A further advantage of one or more embodiments provided by the present disclosure is to detect coughs without involving subjective judgment.

Yet another advantage of one or more embodiments provided by the present disclosure is a cough detection system that can be used in any patient population, including healthy individuals.

Another advantage of one or more embodiments provided by the present disclosure is a cough detection system having operation that is not affected by ambient noise, therefore suitable for day-to-day monitoring in noisy environments, and having simplicity by using only a single accelerometer, and thus the system is usable in a variety of applications such as cough frequency monitoring during sleep studies and veterinary medicine applications.

Additional features and advantages are described herein, and will be apparent from, the following Detailed Description and the Figures.

### BRIEF DESCRIPTION OF THE FIGURES

FIG. 1 is diagram showing the location and orientation of a dual-axis accelerometer sensor on a human's neck.

FIG. 2 is a schematic diagram of an embodiment of a cough detection device in operation.

FIG. 3 is a flowchart of an embodiment of a method according to the present disclosure.

FIG. 4 shows graphs of A-P and S-I signals containing three swallows (dotted black rectangles) and one involuntary cough (solid red rectangles) in Example 1 disclosed herein.

FIGS. 5A-5D are graphs comparing voluntary cough vs. artifact accuracy between pairs of classifiers and feature reduction algorithms from Example 1 disclosed herein (elastic net does not converge for feature sizes less than four and hence the incomplete trend for some pairs).

FIG. 6 is the Wilcoxon ranksum p-value heat-map for voluntary cough vs. non-cough artifacts (N/A: Not Applicable and N/S: Not Significant) from Example 1 disclosed herein.

FIG. 7 are graphs showing a participant's voluntary coughs (solid red) and swallows (dotted black), with A-P and S-I signals in the top panels, and 2D trajectories for swallowing (lower left panel) and cough (lower right panel) after smoothing in Example 1 disclosed herein.

FIGS. 8A-8D are graphs comparing involuntary cough vs. artifact accuracy between pairs of classifiers and feature reduction algorithms from Example 1 disclosed herein (elastic net does not converge for feature sizes less than four and hence the incomplete trend for some pairs).

FIG. 9 are graphs showing a participant's involuntary coughs (solid red) and swallows (dotted black), with A-P and S-I signals in the top panels, and 2D trajectories for swallowing (lower left panel) and cough (lower right panel) after smoothing in Example 1 disclosed herein.

FIG. 10 is a table showing the number of participants and boluses (thin consistency) in the study set forth in Example 2 disclosed herein.

FIG. 11 are graphs showing noise-floor annotated A-P and S-I signals of a bolus in the study set forth in Example 2 disclosed herein. The signal portion that is above the noise-floor threshold is marked in light green.

FIG. 12 is a graph showing scalar analysis over different object function penalty values (β) in the study set forth in Example 2 disclosed herein. The vertical line denotes the optimal value of α.

FIG. 13 is a graph showing instance selection on the basis of proximity to the posterior classification probability threshold in the study set forth in Example 2 disclosed herein.

FIG. 14 includes histograms of VFSS-determined and algorithmically estimated bolus lengths for different scalars (α) in the study set forth in Example 2 disclosed herein.

FIG. 15 is a table of a comparison of the classification performance using the proposed instance selection approaches in the study set forth in Example 2 disclosed herein.

FIG. 16 is a box plot of AUC values for classification with instance selection by posterior probability bands for different removal caps in the study set forth in Example 2 disclosed herein. The x-axis labels indicate the removal cap as a % of the test set. The actual number of test cases removed follows in parentheses. The actual width of the probability margin (δ) is shown above the box plots.

FIG. 17 is a graph of PCA components of selected (red) and non-selected (black) instances in the study set forth in Example 2 disclosed herein. The circles denote safe boluses while asterisks denote unsafe boluses.

FIG. 18 is a graph of parallel features of selected and non-selected instances in the study set forth in Example 2 disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. The words “comprise,” “comprises” and “comprising” are to be interpreted inclusively rather than exclusively. Likewise, the terms “include,” “including” and “or” should all be construed to be inclusive, unless such a construction is clearly prohibited from the context. A disclosure of a device “comprising” several components does not require that the components are physically attached to each other in all embodiments.

Nevertheless, the devices disclosed herein may lack any element that is not specifically disclosed. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified.

The term “and/or” used in the context of “X and/or Y” should be interpreted as “X,” or “Y,” or “X and Y.” Where used herein, the terms “example” and “such as,” particularly when followed by a listing of terms, are merely exemplary and illustrative and should not be deemed to be exclusive or comprehensive. Any embodiment disclosed herein can be combined with any other embodiment disclosed herein unless explicitly stated otherwise.

The term “individual,” “subject” or “patient” means any animal, including humans, that could experience coughing. Indeed, every mammalian species studied to date displays a cough reflex or some similar forceful expiratory reflex evoked by airway irritation. Generally, the individual is a human or an avian, bovine, canine, equine, feline, hircine, lupine, murine, ovine or porcine animal. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Preferably, the patient is a mammal, such as a human or a companion animal, e.g., a dog or cat.

The terms “food,” “food product” and “food composition” mean a product or composition that is intended for ingestion by an individual such as a human and provides at least one nutrient to the individual. These terms include beverages. The compositions of the present disclosure, including the many embodiments described herein, can comprise, consist of, or consist essentially of the elements disclosed herein, as well as any additional or optional ingredients, components, or elements described herein or otherwise useful in a diet. As used herein, a “bolus” is a single sip or mouthful of a food.

“Prevention” includes reduction of risk and/or severity of a condition or disorder. The terms “treatment,” “treat,” “attenuate” and “alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder, and include treatment of patients at risk of contracting a disease or suspected to have contracted a disease, as well as patients who are ill or have been diagnosed as suffering from a disease or medical condition. The term does not necessarily imply that a subject is treated until total recovery. These terms also refer to the maintenance and/or promotion of health in an individual not suffering from a disease but who may be susceptible to the development of an unhealthy condition. These terms are also intended to include the potentiation or otherwise enhancement of one or more primary prophylactic or therapeutic measure. The terms “treatment,” “treat,” “attenuate” and “alleviate” are further intended to include the dietary management of a disease or condition or the dietary management for prophylaxis or prevention a disease or condition. A treatment can be patient- or doctor-related.

### EMBODIMENTS

Cervical accelerometry is a non-invasive and non-radiographic assessment technique where the patient wears a dual-axis accelerometer midline, below the laryngeal prominence (commonly known as the Adam's apple). The accelerometer captures epidermal vibrations in the anterior-posterior (AP) and superior-inferior (SI) directions, thus facilitating day-to-day monitoring of pharyngeal vibrations. An aspect of the present disclosure is an algorithmic approach to accurately differentiate coughs from a resting state, swallowing, head movements and speech on the basis of dual-axis accelerometry signals.

An aspect of the present disclosure is a method of processing dual-axis accelerometry signals to classify one or more of the signals as a cough or a non-cough (e.g., a rest state, a swallow, a tongue movement, or speech). Another aspect of the present disclosure is a device that implements one or more steps of the method.

In an embodiment, the method can further comprise diagnosing and/or treating the patient based on the classification of each of the dual-axis accelerometry signals (e.g., determining a clinical assessment of the patient). For example, a patient can be diagnosed as having a medical condition such as asthma, rhinitis, gastro-oesophageal reflux disease, bronchitis and/or dysphagia if the frequency of the coughs exceeds a threshold. Treatment of the patient can be adjusted based at least partially on the classification of each of the dual-axis accelerometry signals.

In some embodiments, the method and the device can be employed in the apparatuses and/or the methods disclosed in U.S. Pat. No. 7,749,177 to Chau et al., the methods and/or the systems disclosed in U.S. Pat. No. 8,267,875 to Chau et al., the systems and/or the methods disclosed in U.S. Pat. No. 9,138,171 to Chau et al., or the methods and/or the devices disclosed in U.S. Pat. App. Publ. No. 2014/0228714 to Chau et al., each of which is incorporated herein by reference in its entirety.

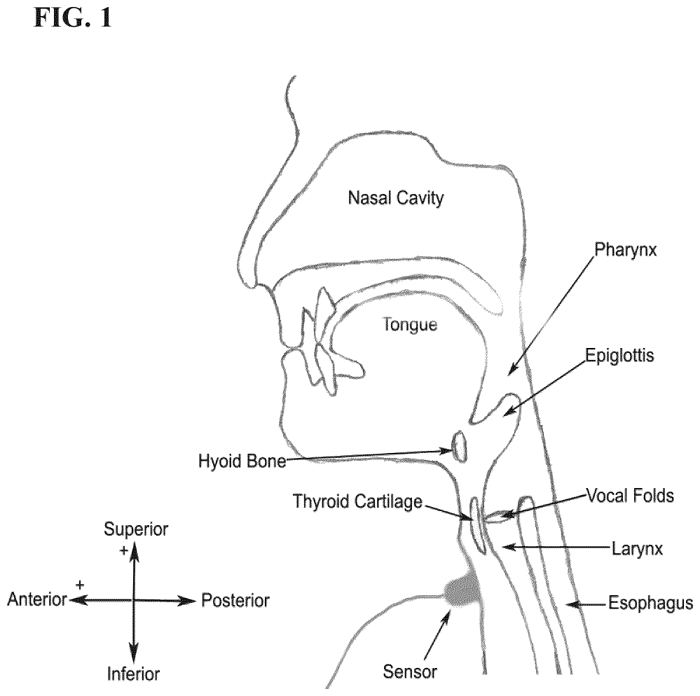

As discussed in greater detail hereafter, the device may include a sensor configured to produce cervical accelerometry signals, preferably a dual axis accelerometer. The sensor may be positioned externally on the neck of a human, preferably anterior to the cricoid cartilage of the neck. A variety of means may be applied to position the sensor and to hold the sensor in such position, for example double-sided tape. Preferably the positioning of the sensor is such that the axes of acceleration are aligned to the anterior-posterior and super-inferior directions, as shown in FIG. 1.

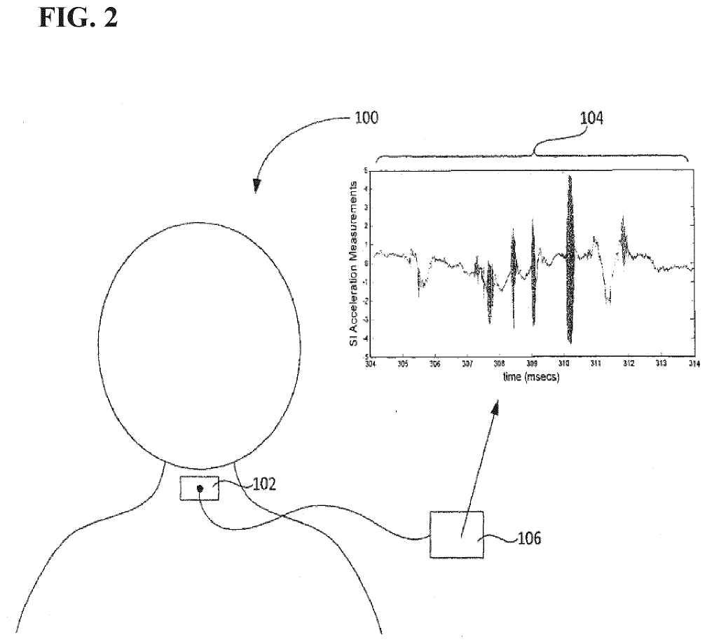

FIG. 2 generally illustrates a non-limiting example of a device 100 for use in cough detection. The device 100 can comprise a sensor 102 (e.g., a dual axis accelerometer) to be attached in a throat area of a candidate for acquiring dual axis accelerometry data and/or signals, for example illustrative S-I acceleration signal 104. Accelerometry data may include, but is not limited to, throat vibration signals acquired along the anterior-posterior axis (A-P) and/or the superior-inferior axis (S-I). The sensor 102 can be any accelerometer known to one of skill in this art, for example a single axis accelerometer (which can be rotated on the patient to obtain dual-axis vibrational data) such as an EMT 25-C single axis accelerometer or a dual axis accelerometer such as an ADXL322 or ADXL327 dual axis accelerometer, and the present disclosure is not limited to a specific embodiment of the sensor 102.

The sensor 102 can be operatively coupled to a processing module 106 configured to process the acquired data for cough detection, for example discrimination between cough and non-cough events such as a rest state, a swallow, a tongue movement, and speech. The processing module 106 can be a distinctly implemented device operatively coupled to the sensor 102 for communication of data thereto, for example, by one or more data communication media such as wires, cables, optical fibers, and the like and/or by one or more wireless data transfer protocols. In some embodiments, the processing module 106 may be implemented integrally with the sensor 102.

Generally, the processing of the dual-axis accelerometry signals comprises representation of the signal segments in meta-features and then classification of each segment based on the meta-features. Preferably the classification is automatic such that no user input is needed for the dual-axis accelerometry signals to be processed and used for classification of the signal.

In a non-limiting embodiment of the methods disclosed herein, dual-axis accelerometry data for both the S-I axis and the A-P axis is acquired or provided, for example dual-axis accelerometry data from the sensor 102. In some embodiments, the dual-axis accelerometry data for both the S-I axis and the A-P axis can be acquired or provided a time period that is at least 10 minutes, preferably at least 30 minutes, more preferably at least 45 minutes, most preferably at least one hour, and in some embodiments at least two, three or four hours). Preferably the method is performed without using a microphone, a video recorder, or another accelerometer, i.e., the dual-axis accelerometry data is acquired without using a microphone, a video recorder, or another accelerometer during the time period.

The dual-axis accelerometry data can optionally be processed to condition the accelerometry data and thus facilitate further processing thereof. For example, the dual-axis accelerometry data may be filtered, denoised, and/or processed for signal artifact removal (“preprocessed data”). In an embodiment, the dual-axis accelerometry data is subjected to one or more of de-noising, head movement suppression, or high frequency noise filtering (e.g., wavelet packet decomposition).

The accelerometry data (either raw or preprocessed) can then be automatically or manually segmented into distinct events. Preferably the accelerometry data is automatically segmented. In an embodiment, the segmentation is automatic and energy-based. In another embodiment, the accelerometry data is automatically segmented as disclosed in U.S. Pat. No. 8,267,875 to Chau et al., the entirety of which is incorporated herein by reference as noted above. For example, the automatic segmentation can comprise applying fuzzy c-means optimization to the data determine the time boundaries for each of the cough and non-cough segments. Additionally or alternatively, manual segmentation may be applied, for example by visual inspection of the data. The methods disclosed herein are not limited to a specific process of segmentation, and the process of segmentation can be any segmentation process known to one skilled in this art.

Then meta-feature based representation of the signals is performed. For example, one or more time features, frequency features, time-frequency features, or information-theoretic features for each segment (i.e., cough, speech, swallow, tongue movement, rest) can be computed from the A-P and S-I axes separately. Non-limiting examples of suitable time domain features include: mean, mean absolute deviation, median, variance, skewness, kurtosis, and memory. Non-limiting examples of suitable information-theoretic domain features include entropy and entropy rate. Non-limiting examples of suitable frequency domain features include peak frequency, bandwidth, Lempel-Ziv complexity, and centroid frequency. Non-limiting examples of suitable time-frequency domain features include maximum energy, wave energy, and discrete wavelet transform (DWT) coefficients.

The meta-feature representation of the dual-axis accelerometry signals can then be used as the input along with respective labels in subsequent feature-selection and/or classification. Preferably a subset of the meta-features may be selected as salient meta-features for classification, preferably predetermined salient meta-features identified by analysis of previous data.

Accordingly, where the device has been configured to operate from a reduced feature set, such as described above, this reduced feature set will be characterized by a predefined feature subset or feature reduction criteria. For example, the meta-features preferably comprise at least one of (i) mean S-I, (ii) Lempel-Ziv complexity S-I, (iii) maximum energy A-P, (iv) variance A-P, and (v) skewness A-P. In such an embodiment, the meta-features can be any number of these features (i)-(v), for example one, two, three, four or even all five of these features, and optionally with one or more of the other features.

Then the salient meta-features can be used to classify segments of the dual-axis accelerometry signals (e.g., each of the segments not removed by pre-processing) as a cough or a rest state and/or as a cough or a non-cough (i.e., rest state, swallow, tongue movement, or speech). Preferably an artificial neural network (ANN) and/or a support vector machine (SVM) is applied as a classification algorithm to the salient meta-features of the segment to classify the segment.

The classification can be used to output for a user of the device 100, such as a clinician or a patient. For example, the processing module 106 and/or a device associated with the processing module 106 can comprise a display that identifies the classification using images such as text, icons, colors, lights turned on and off, and the like. Alternatively or additionally, the processing module 106 and/or a device associated with the processing module 106 can comprise a speaker that identifies classification using auditory signals. The present disclosure is not limited to a specific embodiment of the output, and the output can be any means by which the classification of the segment is identified to a user of the device 100.

The output may then be utilized in screening/diagnosing the tested candidate and providing appropriate treatment, further testing, and/or proposed dietary or other related restrictions thereto until further assessment and/or treatment may be applied. For example, adjustments to feedings can be based on changing consistency or type of food and/or the size and/or frequency of mouthfuls being offered to the patient.

In some embodiments, the method can optionally comprise a validation subroutine in which a data set representative is processed such that each data set ultimately experiences the preprocessing, feature extraction and classification disclosed herein. After all events have been classified and validated, output criteria may be generated for future classification without necessarily applying further validation to the classification criteria. Alternatively, routine validation may be implemented to either refine the statistical significance of classification criteria, or again as a measure to accommodate specific equipment and/or protocol changes (e.g. recalibration of specific equipment, for example, upon replacing accelerometer with same or different accelerometer type/model, changing operating conditions, new processing modules such as further preprocessing subroutines, artifact removal, additional feature extraction/reduction, etc.).

Another aspect of the present disclosure is a method of treating dysphagia. The method of treating dysphagia comprises using any embodiment of the device 100 disclosed herein and/or performing any embodiment of the method disclosed herein. The method can further comprise adjusting a feeding administered to the patient based on the classification, for example by changing a consistency of the feeding, changing a type of food in the feeding, changing a size of a portion of the feeding administered to the patient, changing a frequency at which portions of the feeding are administered to the patient, or combinations thereof.

In an embodiment, the dysphagia is oral pharyngeal dysphagia associated with a condition selected from the group consisting of cancer, cancer chemotherapy, cancer radiotherapy, surgery for oral cancer, surgery for throat cancer, a stroke, a brain injury, a progressive neuromuscular disease, neurodegenerative diseases, an elderly age of the patient, and combinations thereof. As used herein, an “elderly” human is a person with a chronological age of 65 years or older.

In some embodiments, the method and the devices disclosed herein can use instance selection and/or noise-floor bolus length estimation, for example in methods disclosed by U.S. Pat. No. 9,687,191 entitled “Method and Device for Swallowing Impairment Detection,” incorporated herein by reference in its entirety. For example, instance selection and/or noise-floor bolus length estimation can be employed in a method of classifying the vibrational data (e.g., dual-axis accelerometry data) as indicative of one of normal swallowing and possibly impaired swallowing, preferably by classifying one or more swallowing events as indicative of a safe event or an unsafe event. Non-limiting examples of instance selection and noise-floor bolus length estimation are set forth in Example 2 later herein.

Instance selection refers to a family of methods in machine learning that aims to reduce the volume of a given data set to accelerate the training and testing processes while maintaining or surpassing the classification accuracies obtained with the full data set. In general, instance selection algorithms extract a subset of instances from data sets that are suspected of containing ambiguous, superfluous, or noisy data points. The intent is that the extracted subset optimizes classification performance. Ambiguous data points are the instances with classification posteriors close to the classification threshold, while superfluous data points bring no additional value to classification and noisy data points lead to false classification predictions. The choice of instance selection algorithms is problem-specific and no one algorithm is superior over others in all contexts.

Instance selection algorithms can be categorized according to the process of deriving the data subset (i.e. incremental, decremental, batch, mixed, and fixed), the type of discarded instances (i.e., boundary, central, or both), and the selection criterion (i.e., classification performance or feature values). Based on the process of deriving the data subset, instance selection algorithms can be organized into five categories:

Incremental: Instance selection begins with an empty subset and incrementally adds data points by analyzing the instances in the training set.

Decremental: The decremental algorithm begins with the entire training set and removes data points that are suspected of being unnecessary or superfluous; these data points meet the predefined selection criterion.

Batch: The batch instance selection algorithm does not remove instances until all data points have been analyzed. Instances that meet the selection criterion are marked but not removed until all data points have been considered, at which time, all the marked instances are discarded.

Mixed: Mixed instance selection starts with a preselected subset of data points and either adds instances to or removes instances from this subset.

Fixed: Fixed instance selection algorithms constitute a subfamily of the mixed algorithm, where a predetermined subset size is maintained while adding instances to or removing instances from the subset.

Instance selection algorithms can also be classified according to the type of discarded data, namely, points from the decision boundary, “central” points within the boundaries, or combinations thereof:

Condensation: These methods retain data points at the border among classes while selecting central (internal) instances for removal. They argue that the instances closer to the decision boundary play a key role in the classification process while the central data points have relatively little effect on classification performance. Although training accuracy may be preserved with this scheme, the overall test accuracies are often negatively affected. Since the number of central data points are often larger than the border instances, the condensation algorithms generally achieve high rates of data reduction.

Edition: These instance selection algorithms retain the central data points. These methods aim to identify instances that are ambiguous and not well-classified, specifically by their nearest neighbours. However, superfluous central data points that do not necessarily contribute to classification are not removed in these algorithms. The general test accuracies are positively affected while data reduction is modest compared to the condensation instance selection algorithms.

Hybrid: Hybrid instance selection algorithms combine condensation and edition approaches to select both boundary and central instances to maintain or improve classification accuracies.

Lastly, instance selection algorithms can be understood in terms of their selection criterion. Wrapper algorithms embed instance selection in the process of classifier evaluation. Generally, instances with negligible contribution to model prediction are discarded from the training set. The majority of the wrapper algorithms are based on some measure of misclassification of the instances. In contrast, filter-based instance selection rejects instances based on a selection criterion which is independent of the training algorithm but usually relating to the feature values of the instances. The filter approaches either find representative instances from different subspaces of the data set, or base selection on the similarities between pairs of instances.

The instance selection preferably comprises a wrapper approach in which the classification posterior threshold is deployed in a selection criterion, and an instance is selected for removal if the corresponding classification posterior falls within the vicinity of the tuned threshold. Regarding noise-floor bolus length estimation, preferably this process comprises estimating the onset and offset of the bolus signals based on the noise-floor distribution of both the A-P and S-I channels. These processes can achieve improved bolus-level AUC.

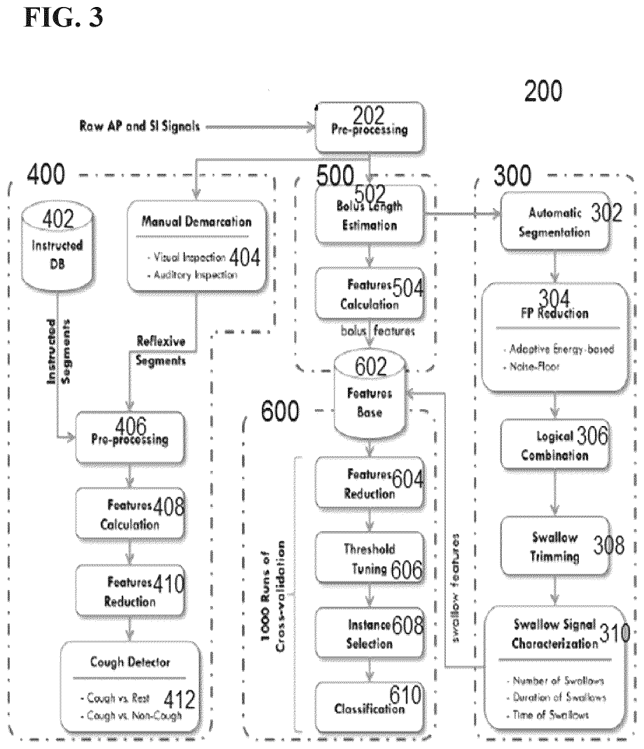

FIG. 3 generally illustrates a preferred embodiment of a method 200 that can be performed by the device 100 according to the present disclosure. The method 200 can comprise the device 100 performing one or more of a pre-processing step 202, a swallow-level analysis process 300, an automatic cough identification process 400 that can discriminate between cough and non-cough artefacts (e.g., for both instructed and reflexive coughs), a bolus length estimation process 500, and a classification process 600.

The classification process 600 can use bolus-level features from the bolus length estimation process 500 and/or swallow-level features from the swallow-level analysis process 300 to provide high sensitivity and specificity discrimination between safe and unsafe swallowing (e.g., in a patient with dysphagia).

For example, the pre-processing step 202 can comprise the device 100 performing one or more of de-noising (e.g., 10-level wavelet decomposition with Daubechies-8 mother wavelets), head movement removal (e.g., B-spline approximation of low frequency (<5 Hz) signal components), speech removal (e.g., eliminating signal segments with periodic behaviors as detected by pitch tracking), or suppression of high frequency noise (e.g., wavelet packet decomposition with a 4-level discrete Meyer wavelet and Shannon entropy).

In a preferred embodiment, the swallow-level analysis process 300 is performed by the device 100 as disclosed in WO 2017/137844 entitled “Signal Trimming and False Positive Reduction of Post-Segmentation Swallowing Accelerometry Data” to Mohammadi et al., the entirety of which is incorporated by reference. For example, the swallow-level analysis process 300 can comprise one or more of automatic segmentation on the dual-axis accelerometry data at Step 302, false positive reduction at Step 304, logical combination at Step 306, swallow trimming at Step 308, or swallow signal characterization at Step 310.

Automatic segmentation on the dual-axis accelerometry data at Step 302 can comprise applying a sequential fuzzy c-means algorithm to the segmented dual-axis accelerometry data.

False positive reduction at Step 304 can use one or both of energy-based false positive reduction or noise floor-based false positive reduction. For example, the energy and noise-floor false positive reduction methods can be applied in parallel on segmented, pre-processed data; and candidate segments identified as valid by at least one of the two false positive reduction methods can be admitted in the logical combination at Step 306.

Swallow trimming at Step 308 can trim the data so that it includes only the portion of the signal corresponding to the physiological vibrations associated with swallowing while excluding the pre- and post-swallow signal fluctuations. For example, the location of the peak amplitude can be found, overlapping windows of size w can be shifted to the left and to the right of the peak by increments of size s, and the energy difference can be calculated within each window. Bilaterally, windowed segments with energy difference below the threshold can be removed from the candidate swallow segment. In an embodiment, this technique employs a kernel density estimation-based algorithm.

Swallow signal characterization at Step 310 can comprise characterization of the dual-axis accelerometry data that has been subjected to segmentation, trimming and false positive reduction. For example, the swallow signal characterization can determine a number of swallows, a duration of swallows, and/or a time of swallows and can identify swallow-level features that can then be subjected to the classification process 600.

The automatic cough identification process 400 can comprise analysis of one or more data sets of dual-axis accelerometry signals by the device 100, and a non-limiting embodiment of the automatic cough identification process 400 is set forth in Example 1 later herein. For example, an “instructed” data set can be provided at Step 402, and/or a “reflexive” data set can be provided at Step 404, and preferably at least one of these data sets comprises data from the pre-processing at Step 202. Additionally or alternatively, the data can optionally be pre-processed at Step 406.

At Step 408, meta-features of the data can be represented, preferably from the A-P signals and the S-I signals separately. Non-limiting examples of such meta-features include temporal, time-frequency, frequency, information-theoretic features for each segment (i.e., cough, speech, swallow, rest) for one or both of the A-P data or the S-I data. Optionally, one or more of mutual information, cross-entropy rate, and cross-correlation between the corresponding A-P and S-I signals can be calculated.

At Step 410, these meta-features can be reduced to a set of salient meta-features, for example meta-features identified as salient by one or more of binary genetic algorithm (BGA), filter-based feature selection, elastic net, or principal component analysis (PCA). Non-limiting examples of salient meta-features include mean S-I, Lempel-Ziv S-I, maximum energy A-P, variance A-P, and skewness A-P. Additionally or alternatively, the device 100 can identify salient features from the information theoretic domain (e.g., entropy and entropy rate) and/or the combination of two axis (e.g., mutual information and cross-correlation).

At Step 412, the device can use the salient meta-features to classify cough segments versus rest states and artefacts, for example by artificial neural network (ANN) or support vector machines (SVM).

The bolus length estimation process 500 and the classification process 600 can comprise analysis of dual-axis accelerometry signals by the device 100, for example data from the pre-processing at Step 202; and a non-limiting embodiment of the bolus length estimation process 500 and the classification process 600 is set forth in Example 2 later herein. For example, at Step 502, the device 100 can perform bolus length estimation, preferably by noise-floor bolus length estimation to estimate the onset and offset of the bolus signals based on the noise-floor distribution of both the A-P and S-I channels.

At Step 504, the device 100 can perform feature selection and extraction, such as by calculation of time, frequency, time-frequency, information theoretic domain features for both A-P and S-I axis and optionally channel combination features as well, and preferably at both bolus- and swallow-level. These features can be provided at Step 602, and a reduced feature set can be identified at Step 604, for example by applying the elastic net as a regularized binary logistic regression used to select a subset of features.

At Step 606, the device 100 can perform threshold tuning, for example by calculating a receiver operating characteristic (ROC) curve using the posteriors of a training data set. At Step 608, the device 100 can perform instance selection to identify and remove uncertain boluses from the dual-axis accelerometry signals. A preferred embodiment of the instance selection employs the classification probability threshold band, e.g., an instance is selected for removal if the corresponding classification posterior falls within the vicinity of the tuned threshold. At Step 610, the device 100 can perform classification to determine a safe or unsafe swallow, for example by applying a linear Discriminant Analysis (LDA) classifier, e.g., an LDA classifier evaluated over 1000 runs of a random hold-out cross-validation test. The device 100 preferably outputs the classification, e.g., by a visual output, such as text, lights, or icons, and/or by an audio output.

### EXAMPLES

The following experimental examples present scientific data developing and supporting an embodiment of the automatic framework for automatic detection of cough and non-cough events, using dual-axis accelerometry signals from a single accelerometer on the patient's neck, as disclosed herein.

### Example 1

### Methodology

The proposed framework that was tested included pre-processing to remove noise and head movements from the acceleration signals. Meta-feature-based representation of the pre-processed signals was then computed followed by feature selection/extraction to identify the most salient features. The salient features were then classified over ten runs of 5-fold cross-validation. The following sections elaborate upon the tested framework in detail.

### Pre-Processing

Pre-processing included de-noising (Sejdić et al., “A procedure for denoising dual-axis swallowing accelerometry signals,” Physiol. Meas. 31:N1-N9 ((2010)) and head movement suppression (Sejdić et al., “A method for removal of low frequency components associated with head movements from dual-axis swallowing accelerometry signals,” PloS ONE 7(3) (2012) e33464; Sejdić et al., “The effects of head movement on dual-axis cervical accelerometry signals,” BMC Res. Notes. 3:269 (2010)). Additionally, high frequency noise was filtered by wavelet packet decomposition using a 4-level discrete Meyer wavelet and Shannon entropy (Mohammadi et al., “Post-segmentation swallowing accelerometry signal trimming and false positive reduction,” IEEE Signal Processing Letters 23(9):1221-1225 (2016); H. Mohammadi, and T. Chau, “Signal trimming and false positive reduction of post-segmentation swallowing accelerometry data”, U.S. patent application Ser. No. 62/292,995 incorporated by reference in its entirety).

### Meta-Feature-Based Representation of Signals

Time, frequency, information-theoretic features for each segment (i.e., cough, speech, swallow, rest) were computed from the A-P and S-I axes separately. For salient feature identification, three selection algorithms to determine parsimonious and discriminatory feature vectors were considered: BGA (binary genetic algorithm) (Mitchell, “An introduction to genetic algorithms,” MIT press, 1998), elastic net (Friedman et al., “Regularization paths for generalized linear models via coordinate descent,” J. Stat. Softw. 33 (1):1-22 (2010)) and filter-based feature selection (Koutroumbas et al., “Pattern Recognition,” 2nd Edition, Academic Press An imprint of Elsevier Science (2003)). Additionally, a reduced feature set was also derived via principal component analysis (PCA).

To invoke GA-based feature selection, candidate feature vectors were coded as a chromosome of Boolean values, each gene indicating whether the corresponding feature is selected. A population size of fifty was selected along with a tournament size of two. Optimization proceeded for a maximum of 100 generations. Crossover and mutation rates of 0.8 and 0.1 were selected respectively. Additionally, in order to keep the best solutions in the population pool, elitism of size two was selected. The entire optimization was iterated 30 times.

For filter-based feature selection, features were ranked based on their uni-dimensional class separability score (Koutroumbas et al. cited above). The top ranking features were then selected as the reduced feature vector. The top five to thirty features were considered for the subsequent classification experiments.

The elastic net is a regularized binomial logistic regression which is used to select a subset of features. With the elastic-net penalty of Zou & Hastie (2005), a set of 10 equally spaced ridge-LASSO penalty (α) values in the range of [0.1, 1] and 100 values of the penalty parameter λ were tested. A pair of α and λ values yielding the minimum 5-fold cross-validated squared-error on the training data was selected using a generalized binomial logistic regression models toolbox (Qian et al., “Glmnet for matlab 2010”).

In addition to the above feature selection approaches, a reduced set of transformed features was also generated using principal component analysis (PCA) (Malhi et al., “Feature selection for defect classification in machine condition monitoring,” Proc. 20th IEEE Instrumentation Measurement Technology Conf., 1:36-41 (2003)). The components were then sorted in descending order based on their corresponding eigenvalues. Classification was then evaluated using different subsets selected from the top of the sorted components in the inner cross-validation.

### Classification

In order to classify cough segments vs. swallow signals, rest states, and all artifacts, artificial neural networks (ANN) and support vector machines (SVM) were deployed as classification algorithms.

Neural networks with a single hidden layer of twenty units and two output units were implemented. This configuration was selected empirically based on the training performance. The inputs were feature values from the reduced feature subsets described above. Networks were trained using Bayesian regularized back-propagation with a mean-squared error criterion function and evaluated via five-fold cross-validation with a 80-20 split into training and validation on the training folds.

A support vector machine with a radial basis function (RBF) kernel with scaling factor of size two was deployed (Duda et al., “Pattern Recognition,” Wiley-Interscience, New York (2001)).

### Validation

To validate the proposed cough detection system, comparisons between pairs of feature selection and classification approaches (e.g., elastic net+SVM) were conducted based on classification performance and model complexity such as number of features. The comparison is conducted for a feature set size ranging from 1 to 35 (i.e., entire feature set).

Feature selection and classifier pairings were evaluated using ten runs of five-fold cross-validation. The model performance was evaluated based on the mean±standard deviation of the pair's accuracy, as well as true positive and true negative rates over ten runs of five-fold cross-validation of the test cases. In each run, the data set was divided into five folds. Each fold was considered as the test set while the feature selection and classifier pair was trained using the remaining four folds and blind to the test cases. This process was repeated ten times.

The elastic net and SVM hyper-parameter (RBF variance) and SVM slack parameter were tuned using the training data set based on inner cross-validation. The inner cross-validation accuracy values of different pairs were evaluated using the Wilcoxon ranksum test.

### Experimental Setup

Two different data sets of dual-axis accelerometry signals (herein referred to as the ‘voluntary’ and ‘involuntary’ cough data sets) were used to validate the cough detection algorithm. Fifteen subjects participated in the voluntary cough data collection. Each participant attended two data collection sessions, each lasting approximately 45 minutes. The protocol was approved by the research ethics board of the participating hospital. Each participant provided written, informed consent.

The first session consisted of only tongue motions (tongue protruding out of the mouth with lips pursed, tongue contacting the inside of the left and right cheeks separately, and tongue at rest). The second session comprised coughing, swallowing water, and saying “on” or “off” out loud. Prior to data collection in each session, the experimenter demonstrated the required tasks and provided participants with five minutes to practice the tasks. Within each session, participants were cued to perform the tasks in a pseudo-random order through a LabVIEW interface. Participants were instructed to perform the task within the 4 seconds immediately following the presentation of each cue. Each task was repeated 20 times for every participant. In total, 300 examples of each task were obtained (15 participants×20 examples of each task/participant). The experimenter noted when the participant performed the incorrect task. The data set thus included accelerometry signals pertaining to tongue movements, coughs, swallows and speech. All signals were trimmed automatically by identifying the one second segment with maximum energy within the 4 second recording. In particular, the trimmed signal was derived by centering a one second window around the location of the signal peak in the maximum energy segment.

Involuntary reflexive coughs were derived from a previously reported dataset (Mohammadi et al., “Post-segmentation swallowing accelerometry signal trimming and false positive reduction,” IEEE Signal Processing Letters 23(9):1221-1225 (2016), cited above). These coughs are associated with swallowing activity, reflecting aspiration events, as opposed to coughs elicited in a cough reflex test (where an irritant like citric acid is infused through a nebulizer to observe the expected cough reflex response).

Dual-axis accelerometry signals were collected from 196 consenting adults living with the effects of stroke or brain injury, or with otherwise unrelated suspicion of dysphagia. Each participant performed a series of 6 discrete sips of thin liquid barium (Bracco Varibar Thin Liquid Barium, diluted to a 20% w/v concentration).

Segments of the accelerometry signals were manually annotated with the labels listed in Table II, using a graphical user interface (GUI) designed in MATLAB that enabled simultaneous visual and aural review of the signals. The GUI enabled marking the start and end times of different events. Through this procedure, a total of 51 coughs (average duration 862.61±536.1 ms) were identified. To facilitate the development of a cough detector, 45 swallow segments (average duration 1198.17±493.6 ms) were further extracted from the signals containing the identified coughs. Additionally, 51 rest segments were extracted from the first ten seconds of recorded data prior to swallowing task commencement. In particular, for a given cough, the pre-task signal segment of the same duration and minimum energy was chosen as the corresponding rest segment. Rest segments were only selected from recordings containing at least one cough segment.

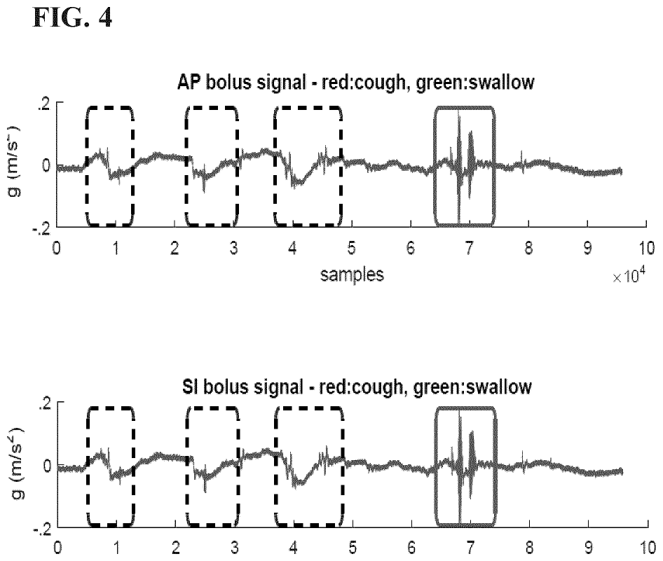

FIG. 4 exemplifies manually annotated coughs and swallows for a participant in the involuntary cough data set. This recording contained three swallows, outlined by the dotted black rectangles, and one cough event, indicated by the solid red rectangles.

### Results

When discriminating between voluntary cough and rest state, SVM and BGA resulted in a high accuracy of 99.26±0.12% with TPR and TNR of 99.96±0.16% and 98.6±0.15%, respectively.

For the involuntary data set, an accuracy of 90±13.9% was achieved with TPR and TNR of 100±0% and 95±6.9%, respectively, for involuntary cough and rest state, using SVM and elastic net.

A more complex classification problem is to discriminate between cough segments and other non-cough artifacts (combination of swallow, speech, and head movement segments).

For the discrimination between voluntary coughs and non-cough artifacts, SVM and elastic net pairing led the way with TPR, TNR, and accuracy of 91.2±4.8%, 89±5.5%, and 90.2±3.6%, respectively.

The leading classification and feature selection pair for involuntary cough vs. non-cough artifacts was SVM and BGA with TPR, TRN, and accuracy of 80.9±15.8%, 79.8±18.6%, and 80.3±10.5%, respectively.

### Discussion

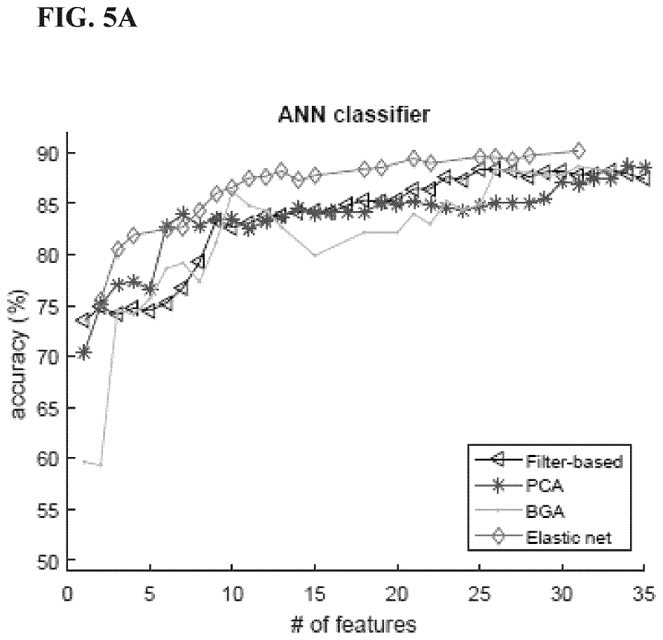

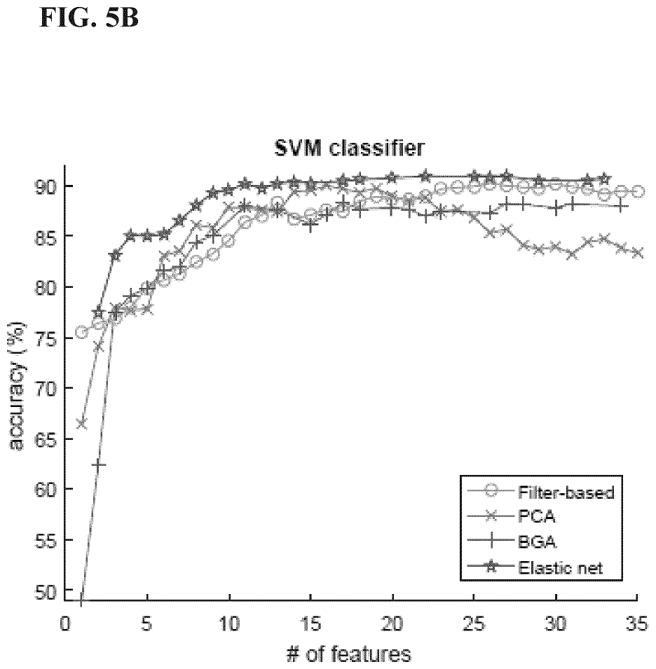

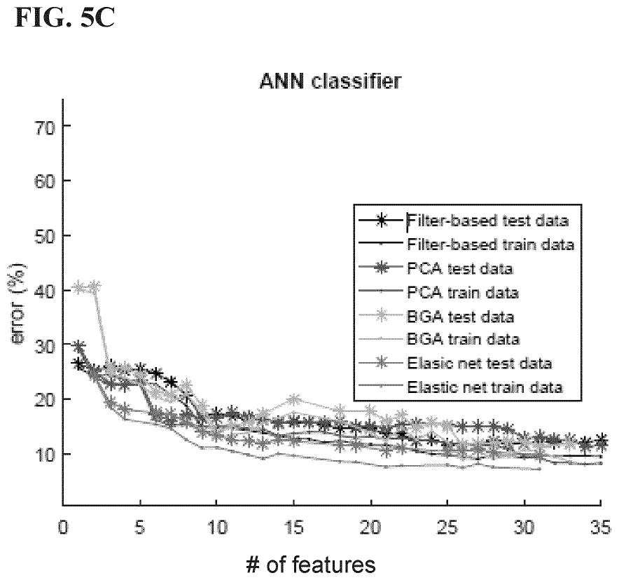

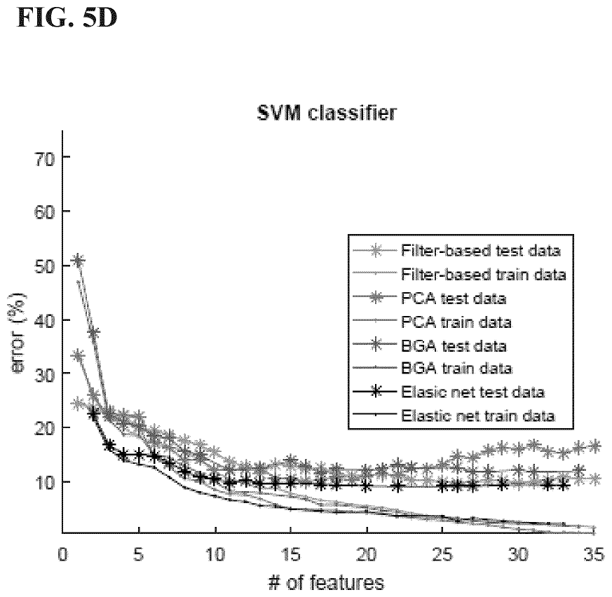

FIGS. 5A-5D demonstrate the accuracy values of discriminating voluntary cough segments from non-cough artifacts. As shown in the bottom right plot, the error rate of the training and test data diverged after 15 features in the case of SVM. This divergence is attributed to over-fitting. For the ANN, as shown in FIG. 5A, the accuracy results saturate after eleven features.

To obtain a fair comparison between the eight classification and feature selection pairs, the Wilcoxon rank sum test was performed on the pairs for different number of feature subsets. The most frequent superior pairs were selected based on the p-value of the rank sum test leading to the optimal salient feature subset size for different pairs.

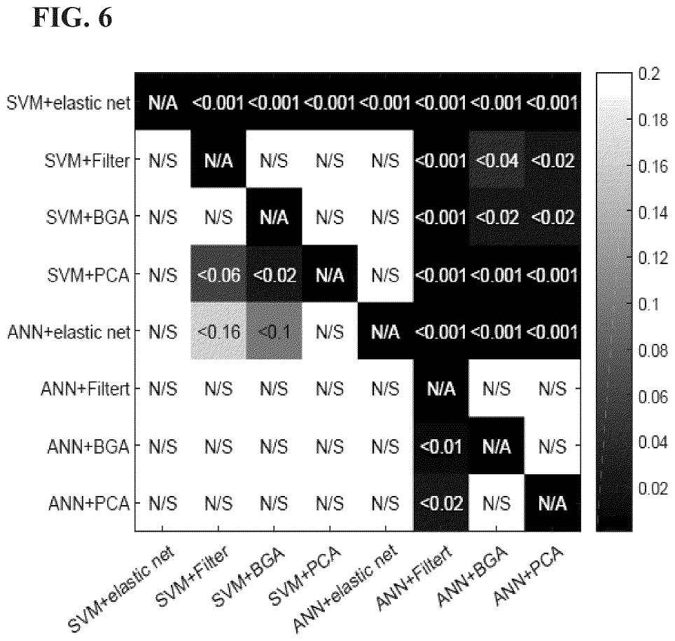

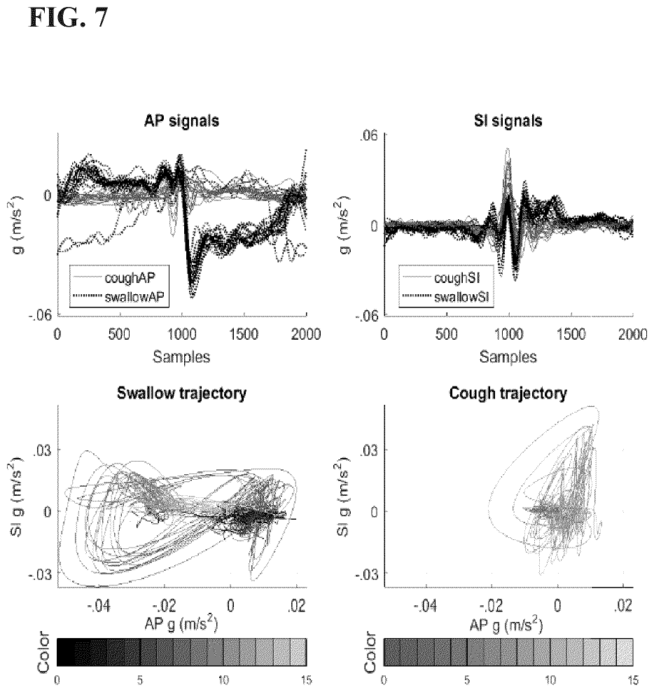

FIG. 6 is a heat-map of the p-values calculated using right-tailed Wilcoxon rank sum test for the optimal number of features of each pair for the voluntary data set. The right-tailed p-values examines whether the algorithm pairs on the y-axis has a greater median compared to the algorithm pairs on the x-axis. FIG. 6 shows that the leading pair is SVM and elastic net (p<0:001). As shown in FIG. 7, trajectories of cough segments appear to be qualitatively more complex than swallowing segments.

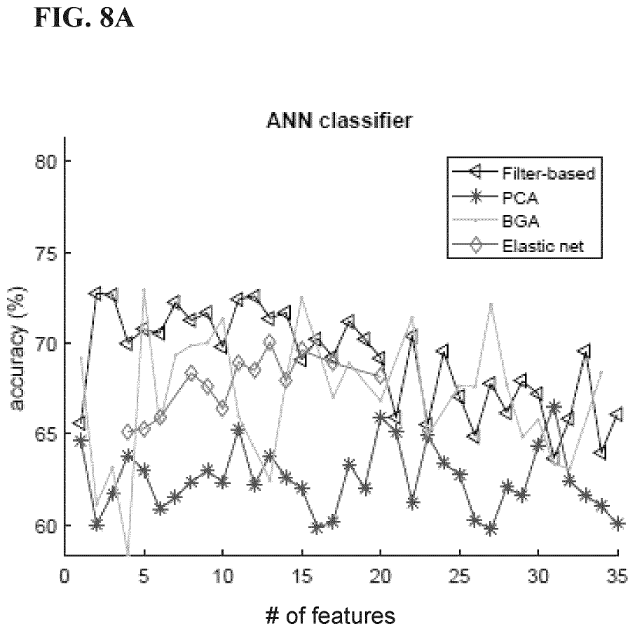

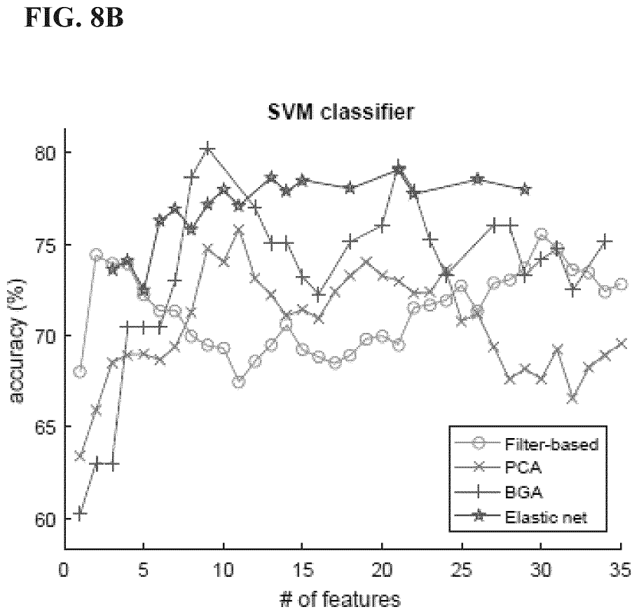

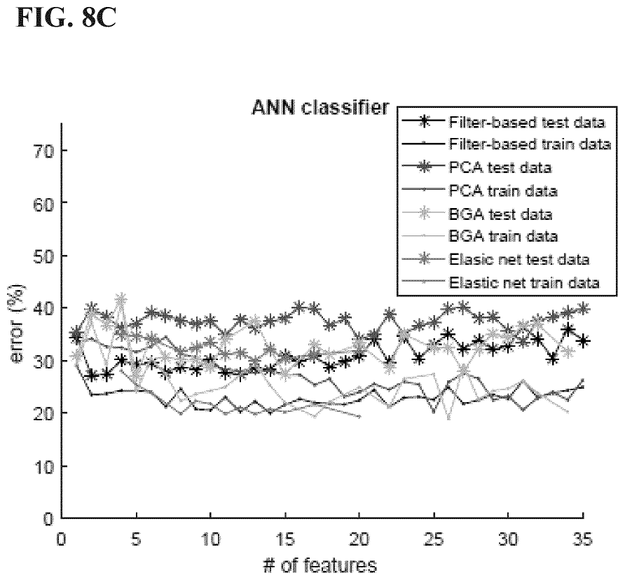

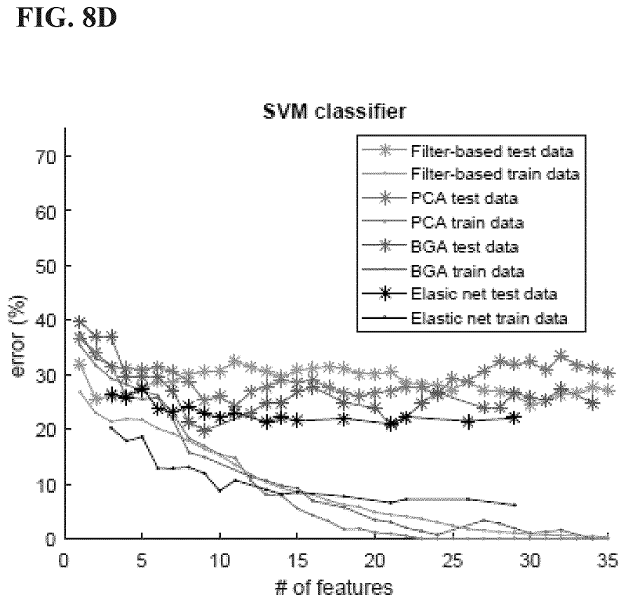

FIGS. 8A-8D present the results of classifying involuntary coughs versus non-cough artifact segments, over different feature subsets using different feature selection/reduction and classification methods. Although elastic net demonstrated a more regular and steady performance over different subsets of features, the leading feature reduction and classification pair is SVM and BGA (p<0:03).

The following five features were selected frequently for both voluntary and involuntary classifications: mean S-I, Lempel-Ziv S-I, maximum energy A-P, variance A-P, and skewness A-P. Evidently, features from both A-P and S-I axis were selected. This finding emphasizes that a dual-axis accelerometer provides more informative signals.

In addition, the unique salient features for the involuntary classification were selected from the information theoretic domain (e.g. entropy and entropy rate) and the combination of two axis (e.g. mutual information and cross-correlation), while the majority of salient features for the voluntary classification were from the time domain (e.g. memory and kurtosis).

The entropy rate characterizes a stochastic process and measures the regularity of the signal and is used in deemed suitable for swallowing accelerometry analysis (Lee et al., “Effects of liquid stimuli on dual-axis swallowing accelerometry signals in a healthy population,” Biomedical Engineering OnLine 9(1):1 (2010), cited above). Entropy and mutual information measure the amount and redundancy of information within the signal, respectively. Additionally, appearance of cross-correlation among salient features shows that the correlation between the two A-P and S-I axis is more distinctive for involuntary signals compared to the voluntary tasks.

The memory of a signal measures the temporal extent of the correlation of the neighboring data samples. The kurtosis of a signal measures the peakedness of the amplitude distribution (Lee et al., “Time and time-frequency characterization of dual-axis swallowing accelerometry signals,” Physiol. Meas. 29(9):1105 (2008), cited above). Selection of these features as top salient features for classification of voluntary signals shows that the time domain features are more distinctive when discriminating voluntary tasks compared to involuntary signals.

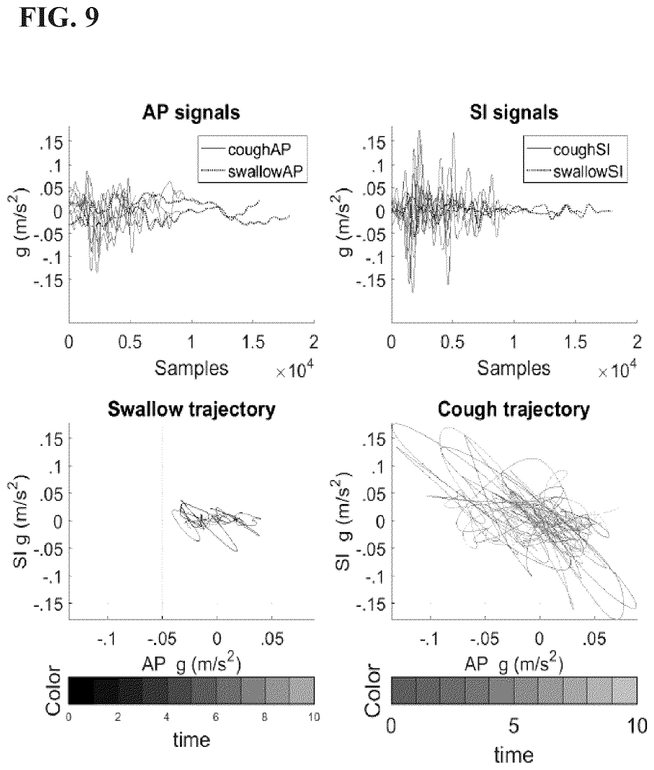

Different salient feature subsets highlights that the voluntary and involuntary signals are different in nature and studies performed based on voluntary signals require more precaution. Additionally, involuntary cough and swallow signal trajectories for a randomly selected participant are shown in FIG. 9. There is no unified pattern recognizable for the cough or the swallow signals. This behavior is evident in all participants, showing both inter- and intra-subject variability.

SVM gave better performance compared to ANN in the majority of comparisons (Wilcoxon ranksum p<0:05). This performance may be due to one of the advantages of SVM classifiers that they find the global minimum, while ANN classifiers may suffer from multiple local minimum solutions (Taylor, “Kernel methods for pattern analysis,” Cambridge university press (2004)). On the other hand, SVM was trained faster than ANN, which makes SVM a more suitable candidate for online analysis and classifications.

One of the advantages of the proposed system is its simplicity, deploying only a single accelerometer. Additionally, the proposed system is not affected by ambient noise, therefore suitable for day to day monitoring in noisy environments. Consequently, potential applications such as cough frequency monitoring during sleep studies and veterinary medicine applications may benefit from this algorithm.

### CONCLUSION

An automatic cough detection and monitoring system discriminated cough accelerometry signals from other artifacts such as rest state, swallowing, head movements, and speech. Both voluntary and involuntary coughs were considered. The proposed system discriminated between coughs and rest state with accuracies of 99.64% and 90% for voluntary and involuntary coughs, respectively. Additionally, the cough segments were discriminated from the non-cough artifacts with accuracy values of 90.2% and 80.3% for voluntary and involuntary data sets.

### Example 2

### Data Set

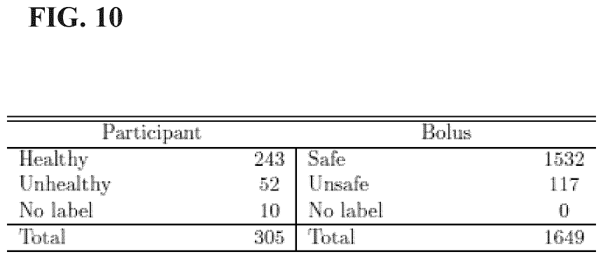

An expanded version of the data reported in Mohammadi et al., “Post-segmentation swallowing accelerometry signal trimming and false positive reduction,” IEEE Signal Processing Letters 23(9):1221-1225 (2016) (cited above) was analyzed. Briefly, acceleration signals were collected from both axes (anterior-posterior (AP) and superior-inferior (SI)) of a dual-axis accelerometer situated on and slightly below the laryngeal prominence (commonly known as the Adam's apple) of participants with suspicion of swallowing difficulties. Acceleration signals were recorded at 10 kHz with 12-bit resolution and filtered in hardware using a passband between 0.1 Hz and 3 kHz. The digitized samples were then stored on a computer with concurrent videouoroscopy for offline analysis. Signals were recorded while patients took 6 sips of thin liquid barium. A sip of barium-coated liquid is referred to as a bolus, which can be ingested in one or multiple swallows. Bolus onset and offset were marked in the accelerometry signals according to expert annotations of the corresponding videouoroscopy recordings. A total of 1,649 usable boluses were identified. A bolus was labeled as unsafe if it contained at least one swallow with a Penetration-Aspiration Scale (PAS) score of 3 or higher while a safe label was given otherwise. For the purpose of this research, only swallows pertaining to thin liquid barium consistency were considered. FIG. 10 summarizes the characteristics of the data set.

### Methodology

### Pre-Processing and Swallow Segmentation

A-P and S-I signals were de-noised using 10-level wavelet decomposition with Daubechies-8 mother wavelets. Signal artefacts relating to head movement were removed by subtracting a B-spline approximation of low frequency (<5 Hz) signal components while vocalizations were suppressed by eliminating signal segments with periodic behaviors, as detected by pitch tracking. Channel-specific normalization was applied to the bivariate bolus signals to scale the signals to [0, 1].

A-P and S-I variance signals were computed by estimating the sample variance within windows of size 200 data points, shifted along each of the A-P and S-I signals with 50% overlap. The swallows were then segmented by subjecting the variance signals to a sequential fuzzy c-means algorithm. The aforementioned segmentation algorithm was too liberal, admitting pre- and post-swallowing activity while also giving rise to non-swallow segments or false positives. A kernel density estimation-based algorithm was used to adaptively trim the swallow segments, while energy and noise floor algorithms reduced the number of false positive swallow segments.

### Feature Selection and Extraction

Time, frequency, time-frequency, information theoretic domain features for both A-P and S-I axis and channel combination features at both bolus- and swallow-level were calculated. The elastic net is a regularized binary logistic regression which is used to select a subset of features. It linearly combines the penalties of the LASSO (Least Absolute Shrinkage and Selection Operator) and ridge regularization methods.

### Noise-Floor Bolus Length Estimation

The majority of the existing studies are dependent on VFSS to demarcate the bolus onset and offset of the acquired acceleration signals. As a result, the existing systems are not completely automated and rely on an external point of reference to segment the signal portions of interest. The proposed noise-floor bolus length estimation reduces the level of VFSS-dependency of the acquired acceleration signals by adding a cushion of 5,000 samples before and after the VFSS annotated boluses and subsequently re-estimating the bolus boundaries. This is possible since the recordings of the accelerometer were continuous. By shifting the VFSS annotated onset to the left and the offset to the right, a more liberal bolus length is selected. The noise-floor algorithm then automatically estimates the bolus length to be as close as possible to the VFSS annotated onset and offsets.

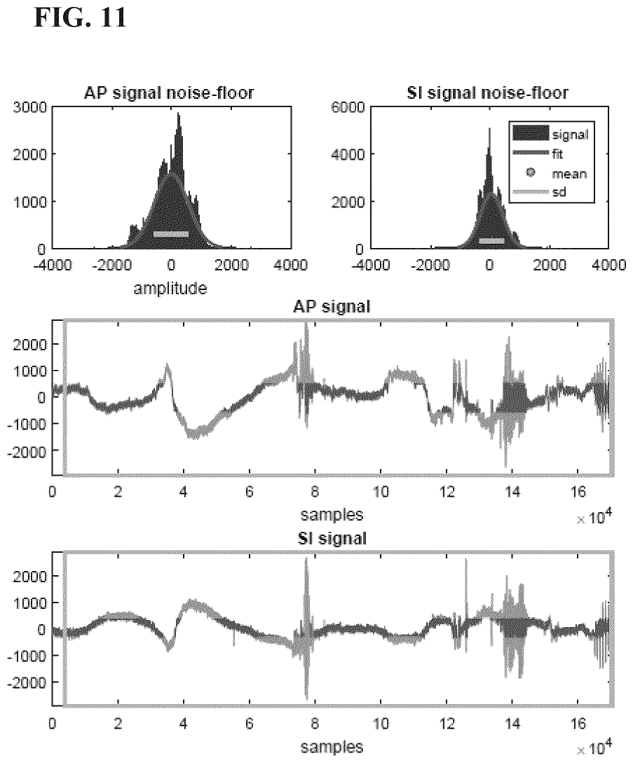

To calculate the noise-floor of the bolus signals, the amplitude histogram of both A-P and S-I channels of the expanded signal were first computed (FIG. 11). After removal of head motions and vocalizations, the remaining noise will generally be of low energy. The range of the noise signal was estimated as α×2σ, where α is a scalar multiplier and σ is initially the bolus signal standard deviation:

$$\text{?}$$

$$\text{?} \text{indicates text missing or illegible when filed}$$

This expression provides an estimate of the range of the noise (i.e., assuming that the noise resided with μ+2ασ and μ−2ασ. The axial thresholds are then determined as:

T^{AP}=α×2σ^{AP} and T^{SI}=α×2σ^{SI}

To estimate the optimum values for A-P and S-I, the following criterion function was considered:

$$\text{?}$$

$$\text{?} \text{indicates text missing or illegible when filed}$$

where δ′₁ and δ′₂ are the new estimated bolus onset and offset, respectively, and δ₁ and δ₂ are the VFSS onset and offset respectively, expressed as a function of the threshold scalar α. The parameter 0≤β<1 is used to tune the objective function. Larger values of β yield more liberal estimates of onsets and offsets, i.e., further away from VFSS values, whereas smaller values of β provides more conservative estimates. The optimal scalar is given by:

$$\text{?}$$

$$\text{?} \text{indicates text missing or illegible when filed}$$

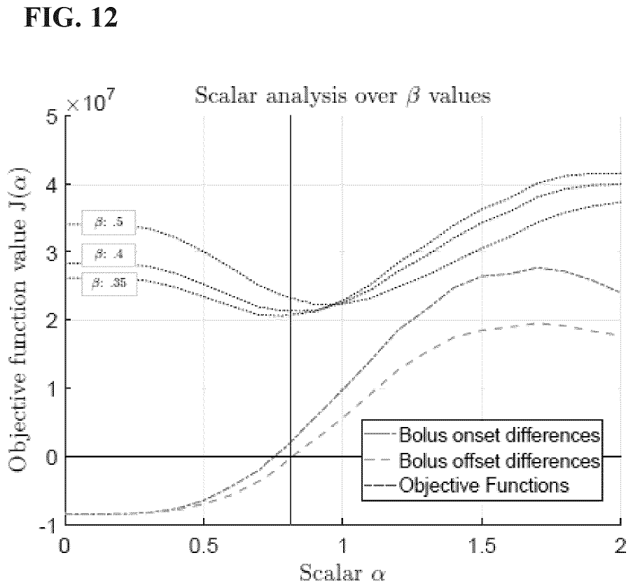

The optimal value of α for the data set under consideration was determined via leave one-out cross-validation with different values of β. The differences between predicted values of bolus onsets and offsets and those determined via VFSS were minimized with α=0:81. For this optimal α, FIG. 12 depicts the objective function values at different values of β. As seen in this figure, a β of 0.35 provided an objective function that yielded the lowest error (i.e., boluses closest in length to those annotated by VFSS) in the neighborhood of the optimal a value. Once α and β were optimized, those values were used in the bolus length estimation algorithm described above in classifier evaluation, i.e., to predict bolus lengths for each training and testing case.

### Instance Selection

To reduce the effect of noisy instances on classification, a filter approach to instance selection was first attempted, and subsequently a posterior probability-guided wrapper approach was proposed.

A simple multidimensional feature-based interquartile-range filter was proposed for instance selection. The 10 most salient features were considered. Let J represent the dimensionality of the feature space and N the total number of instances. Let bᵢ=[f_{i,1}, f_{i,2}, . . . , f_{i,J}] denote a single J-dimensional feature vector corresponding to the i^{th} bolus. Let Q₁=[Q₁₁, Q₁₂, . . . , Q_{1J}] and Q₃=[Q₃₁, Q₃₂, . . . , Q_{3J}] be the lower and upper interquartile values, respectively, for the J features. Let IQR=[IQR₁, IQR₂, . . . , IQR_{J}] denote the interquartile ranges of the J features.

The set of J-dimensional excluded instances Θ is then defined by:

Θ={∀i.bᵢ|bᵢ<Q₁−δ×|IQR∨bᵢ>Q₃+δ×IQR.

1≤i≤.V}

where δ=1.5 in the classical definition of outlying cases.

An alternative, wrapper-based approach to instance selection is to deploy the classification posterior threshold in a selection criterion. A receiver operating characteristic (ROC) curve was calculated using the posteriors of the training data set where each point on this curve results defines a sensitivity and specificity pairing. To account for class imbalance (in this case, minority positive class), the classification posterior threshold was tuned, using only the training set in each cross-validation run, to maximize sensitivity while maintaining 60% classification specificity.

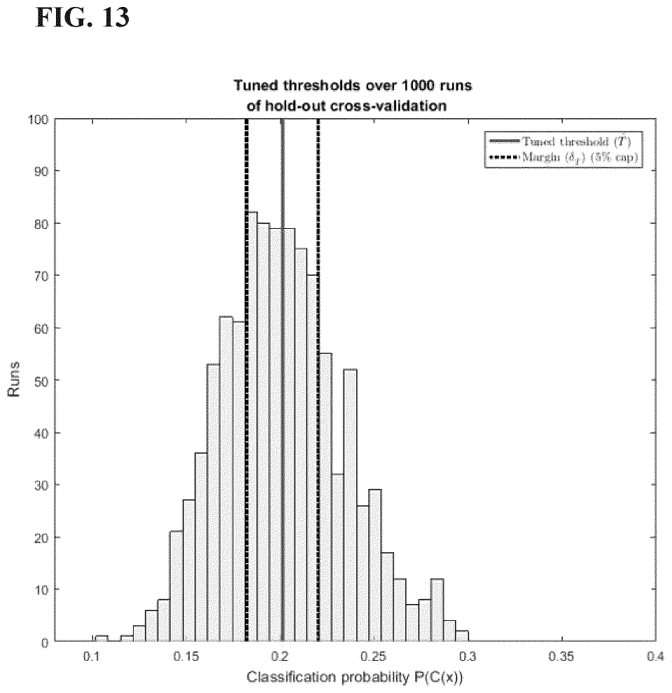

In this approach, an instance was selected for removal if the corresponding classification posterior fell within the vicinity of the tuned threshold. The reasoning is that the uncertainty in the classifier's decision is maximal at the decision threshold and decreases as posterior values depart from the threshold, either increasing in value towards unity or decreasing in value towards zero. In order to limit the number of selected instances, a marginal window was set (FIG. 13). After tuning a classification posterior threshold in each cross-validation run, a probability window of size 0.02 centred around the threshold was considered. The size of this window was then incremented by 0.01 in each direction (above and below the threshold), admitting more instances while not exceeding a selection cap of 5%. This margin along with the tuned threshold was then applied to the test data set. In other words, instances that met the following condition were selected for removal.

\dot{T}−δ_{\dot{T}}<P(C(χ)|X=χ)<\dot{T}+δ_{\dot{T}}

where \dot{T} is the tuned threshold. δ is the margin based on the instance removal cap. P(C(χ)|X=χ) is the posterior probability of instance χ and C(χ)={‘safe’. ‘unsafe’} is the bolus target class label.

### Classification and Evaluation

A Linear Discriminant Analysis (LDA) classifier was evaluated over 1,000 runs of a random hold-out cross-validation test. The entire data set was randomly divided into training and test participants (80% and 20%, respectively) in each run, and the cross-validation runs were completely independent. In each run, a classifier was trained, using only the boluses of the training participants and then tested using the remaining 20% of the participants that were held out. The training and test data sets were selected at participant level, such that the test data set did not contain any boluses from the participants whose data were selected as part of the training data set. Moreover, the classifiers in each run were oblivious to the test and training sets of other runs. Classification performance was assessed in terms of sensitivity, specificity, and area under curve (AUC) across the cross-validation runs. Incidentally, artificial neural network (ANN) and support vector machine (SVM) classifiers were also trained but did not demonstrate any added value in terms of the above classification metrics.

### Results

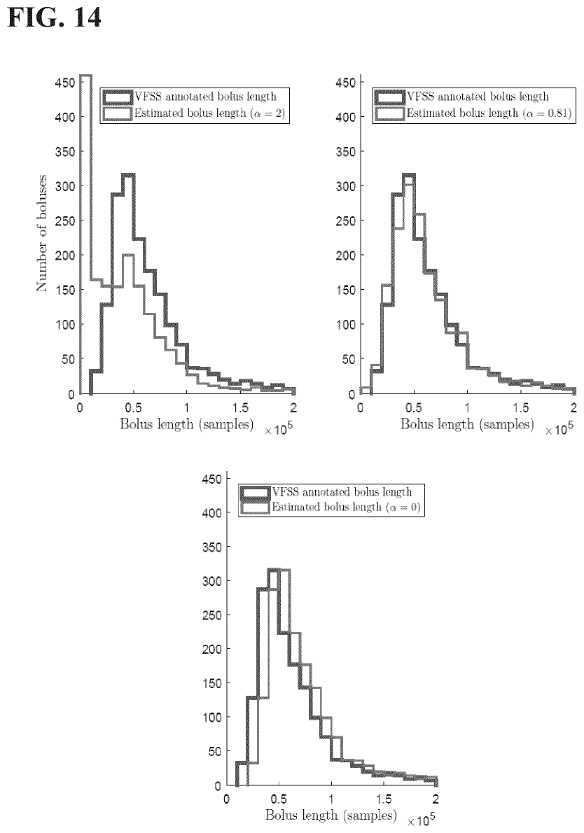

Using the noise-floor bolus length estimation algorithm with the scalar (α) value of 0.81, the performance of the classification system remained unchanged when compared to classification based on VFSS-demarcated boluses. FIG. 14 shows that there is no systematic bias in the length of the boluses before and after application of the noise floor bolus length estimation algorithm using the scalar (α) value of 0.81 (p=0:36, Kolmogorov-Smirnoff test). A kernel density estimate of the VFSS bolus lengths provided the null hypothesis cumulative distribution function against which each distribution of bolus lengths for a given a were tested using the Kolmogorov-Smirnoff goodness-of-fit test.

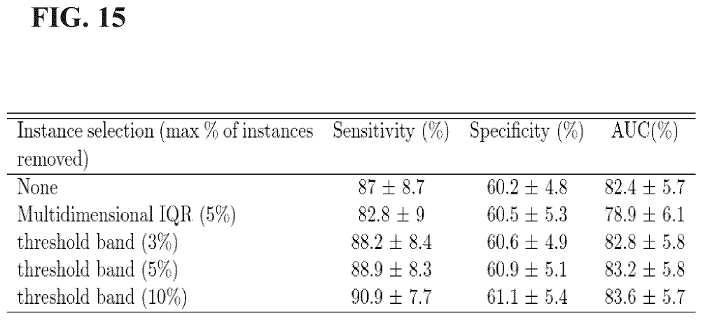

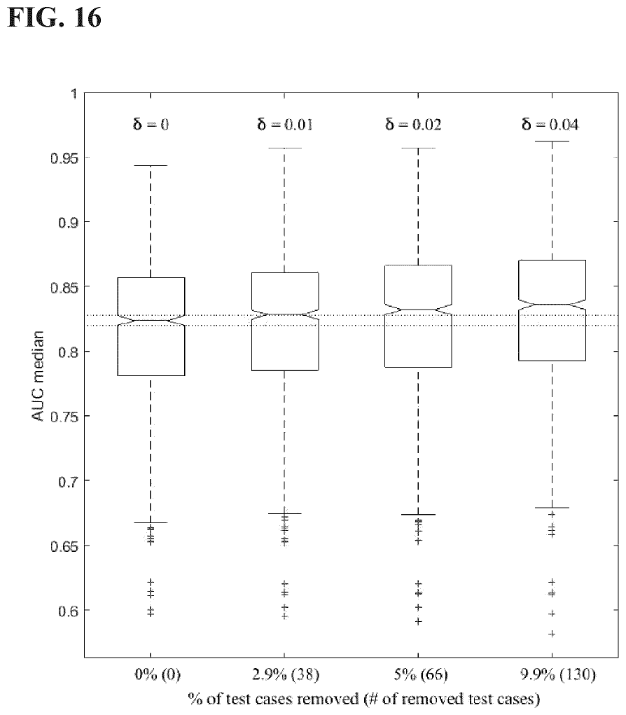

FIG. 15 compares classification performance with and without the different instance selection algorithms after 1,000 runs of hold-out cross-validation. As shown, a maximum AUC of 83.6% was achieved for the discrimination of safe and unsafe boluses of thin consistency. There is an improvement in AUC (p<0:001, Wilcoxon rank sum test) over the no instance selection case when applying the threshold band algorithm with either a 5 or 10% removal cap. This is further elucidated in FIG. 16 where the notches of the box plots for 5 and 10% instance removal do not intersect the notches of the boxplot of the default case.

### Discussion

This example introduced bolus length estimation and instance selection as new elements to swallowing accelerometry classification. The former estimates the onset and offset of the bolus signals, based on the noise-floor distribution of both the A-P and S-I channels, and hence reduces classifier dependency on VFSS-based annotation. This reduced reliance on manual segmentation sets the stage for the development of a standalone, practical device for assessing swallowing safety. Instance selection, on the other hand, objectively identifies instances that diminish classification performance. The aforementioned classification framework achieves improved bolus-level AUC.

Reduced Dependence on VFSS-based determination of bolus of interest: As shown in FIG. 13, larger values of the noise-floor bolus length estimation algorithm scalar (α) forces the algorithm to estimate shorter bolus lengths. Smaller values of on the other hand yields longer boluses. An optimal value of α, which is achieved by minimizing the objective function given in the above-noted equations for T^{AP} and T^{SI}, produced bolus lengths closest to those obtained via VFSS, while maintaining classification performance.

By reducing the dependency on VFSS annotations, a standalone system can eventually be achieved. The addition of the cushion to the beginning and end of the bolus mimics the demarcations one might obtain from operator button presses to bookmark the swallowing activity pertaining to each bolus. The proposed noise floor algorithm then provides an estimate of the bolus boundaries that one might obtain from VFSS review. To our knowledge, all previous swallowing accelerometry studies performed feature calculation, analysis, and classification on the basis of VFSS-demarcated signals, which precludes those algorithms from direct implementation into an independent swallow monitoring system.

Value of Instance Selection: The multidimensional feature-based interquartile-range approach to instance selection discarded boluses with extreme feature values. Since the extreme data points had a defining role in classification training and performance, this approach, although commonly used in the literature, failed to increase the performance of the classifier.

Instance selection using the classification probability threshold band, on the other hand, demonstrated very promising results. This approach leveraged classifier uncertainty as expressed through posterior probabilities. In the cases where the classification probability of the data points were close to the tuned threshold, there was uncertainty in the discrimination between the two classes. By removing instances within the uncertain band enveloping the tuned threshold, the overall performance of the classification algorithm increased significantly, even when only a modest fraction of instances were discarded (5-10%).

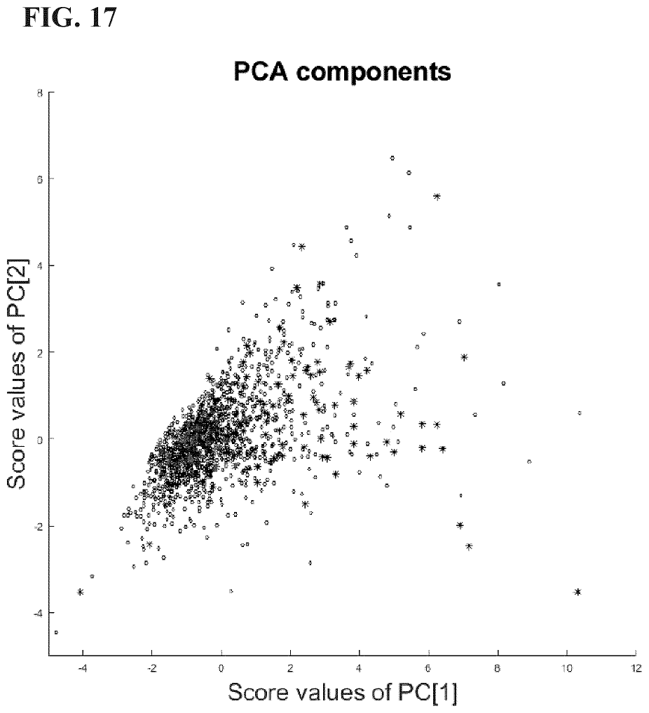

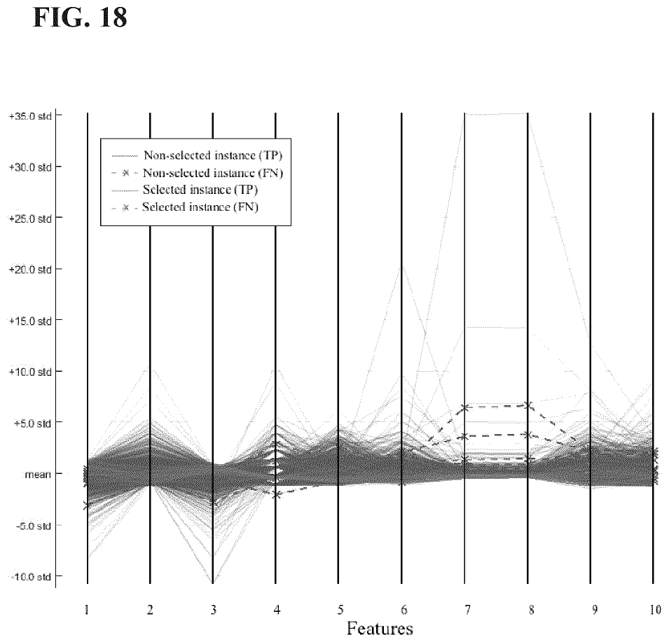

Exploration of Removed Cases: this section investigates the selected instances for the case of the 5% removal cap. Although the classification posterior of the selected instances were marginal (i.e., close to the decision boundary), the feature values of these instances where interior to the feature clusters. FIG. 17 shows the first two components (derived using PCA) of these instances. As shown, the majority of the selected instances reside inside the class clusters. FIG. 18 illustrates the parallel coordinate plot of the 10 salient features for the selected instances, again corroborating the observation that the selected instances are interior to the feature clusters rather than outlying observations.

The origin of the selected instances was as follows: 28.6% were drawn from unhealthy participants while 71.4% came from healthy participants. Notably the original data set was imbalanced with 17.6% and 82.4% of unhealthy and healthy participants, respectively. Despite this class imbalance, the instance selection algorithm disproportionately oversampled the unhealthy participants, suggesting a tendency for indeterminate cases to stem from unhealthy participants. In the original data set, 7% of boluses were unsafe while 92.9% were safe. Of the instances identified by the probability threshold band instance selection algorithm, 4.9% were unsafe boluses and 95.1% were safe boluses. Considering the algorithm's oversampling of unhealthy participants, this latter finding indicates that many safe boluses of unhealthy participants were selected as uncertain. Additionally, 3.4% of the total unsafe boluses and 5.1% of the total safe boluses were selected as uncertain instances. This further emphasizes that most of the selected instances were safe and potentially from unhealthy participants. These safe but uncertain boluses may possess characteristics that are very different from the safe boluses of the healthy participants.

To further investigate the selected instances, a 5-fold cross-validation classification was performed between the selected instances and the remaining (unselected) cases. The selected instances could be discriminated from the rest of the data set with a high accuracy of 98%. This finding confirms that the selected instances exhibit very different signal characteristics from the rest of the data set.

Additionally, the majority of the selected instances were collected from 3 sites (31.52%, 22.42% and 22.42% of instances from sites 1, 4 and 7, respectively). Further investigation of site-specific protocol compliance, as well as inter- and intra-participant variation may provide additional insight into the tendency of uncertain cases to originate from these 3 data collection sites.

Classification Performance: The safe and unsafe bolus-level classification performance achieved in this study is competitive when considering clinical detection rates reported in the literature. According to a recent study, sensitivity and specificity of clinical evaluations are reported to be 39% and 80% respectively, for penetration and 55.6% and 80.5% for aspiration. In other studies, detection sensitivity and specificity have been cited as 88±8% and 50±13%, respectively and 93±21% and 56±20%.

### CONCLUSION

Bolus length estimation and instance selection were introduced as enhancements to swallowing accelerometry classification, on one-hand liberating classification algorithms from manual segmentation of swallows and secondly affording the classifier the freedom to abstain from a decision in the face of uncertainty. Together these enhancements lead to an improvement in AUC in the discrimination between safe and unsafe swallows in a sizable clinical data set.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of identifying a cough, the method comprising:
receiving, on a processing module, dual-axis accelerometry signals obtained by a sensor positioned externally on an anterior-posterior (A-P) axis and a superior-inferior axis (S-I) of the throat of a subject;
representing segments of the dual-axis accelerometry signals as meta-features comprising salient meta-features, the processing module performs the representing of the segments; and
classifying the segments as one of a plurality of classifications comprising at least one classification that is a cough and at least one classification that is a rest state, the processing module performs the classifying based on the salient meta-features.

2. The method of claim 1 wherein, for each of the A-P axis and the S-I axis, at least one of the salient meta-features is selected from the group consisting of time domain characteristics of the accelerometry signals, information theoretic domain characteristics of the accelerometry signals, frequency domain characteristics of the accelerometry signals, and time-frequency domain characteristics of the accelerometry signals.

3. The method of claim 1 wherein at least one of the salient meta-features is selected from the group consisting of mean S-I, Lempel-Ziv complexity S-I, maximum energy A-P, variance A-P, and skewness A-P.

4. The method of claim 1 wherein the classifying of the segments comprises applying at least one of an artificial neural network (ANN) or a support vector machine (SVM) to the salient meta-features.

5. The method of claim 1 wherein the plurality of classifications comprises an additional classification that is at least one non-cough artifact selected from the group consisting of a swallow, a tongue movement, and speech.

6. The method of claim 1 wherein the sensor is a single dual-axis accelerometer, and the method is performed without using a microphone, a video recorder, or another accelerometer.

7. The method of claim 1 comprising pre-processing of the dual-axis accelerometry signals before the representing of the segments of the dual-axis accelerometry signals as the meta-features, the pre-processing comprising at least one step selected from the group consisting of de-noising, head movement suppression, and high frequency noise filtering by wavelet packet decomposition.

8. The method of claim 1 wherein the plurality of classifications comprise at least one classification that is a voluntary cough and at least one classification that is an involuntary cough, and the method comprises discriminating between voluntary cough and involuntary cough.

9. An apparatus comprising:
a sensor configured to be positioned on the throat of a patient and acquire vibrational data for an anterior-posterior axis and a superior-inferior axis; and
a processing module operatively connected to the sensor and configured to represent segments of the dual-axis accelerometry signals as meta-features comprising salient meta-features used by the processing module to classify the segments as one of a plurality of classifications comprising at least one classification that is a cough and at least one classification that is a rest state or a swallow.

10. The apparatus of claim 9 comprising an output component selected from a display, a speaker, and a combination thereof, the processing module configured to use the output component to indicate the classification of the segments visually and/or audibly.

11. The apparatus of claim 9 wherein the processing module is operatively connected to the sensor by at least one of a wired connection or a wireless connection.

12-18. (canceled)

19. A method of classifying a swallow, the method comprising:
receiving, on a processing module, dual-axis accelerometry signals obtained by a sensor positioned externally on an anterior-posterior (A-P) axis and a superior-inferior axis (S-I) of the throat of a subject;
performing at least one enhancement step on the dual-axis accelerometry signals, the at least one enhancement step selected from the group consisting of (i) bolus length estimation on the dual-axis accelerometry signals to identify bolus-level features in the dual-axis accelerometry signals and (ii) instance selection to identify and remove uncertain boluses from the dual-axis accelerometry signals, the processing module performs the at least one enhancement step; and
classifying segments of the dual-axis accelerometry signals as one of a plurality of classifications comprising a first classification and a second classification, the processing module performs the classifying based at least partially on the dual-axis accelerometry signals that have been subjected to the at least one enhancement step.

20. The method of claim 19, wherein ach of the segments is representative of a swallowing event, the first classification is indicative of a safe walling event, and the second classification is indicative of an unsafe swallowing event.

21. The method of claim 20, wherein the swallowing safety impairment is airway invasion at or below the true vocal folds.

22. The method of claim 19, wherein the bolus length estimation comprises noise-floor bolus length estimation.

23. The method of claim 19, wherein the instance selection uses a classification probability threshold band.

24. An apparatus for screening, diagnosing or treating dysphagia, the apparatus comprising:
a sensor configured to be positioned on the throat of a patient and acquire vibrational data for an anterior-posterior axis and a superior-inferior axis; and
a processing module operatively connected to the sensor and configured to perform at least one enhancement step on the vibrational data, the at least one enhancement step selected from the group consisting of (i) bolus length estimation on the vibrational data to identify bolus-level features in the vibrational data and (ii) instance selection to identify and remove uncertain boluses from the vibrational data, the processing module further configured to classify segments of the vibrational data as one of a plurality of classifications comprising a first classification and a second classification based at least partially based on the vibrational data that has been subjected to the at least one enhancement step.
